(19) United States
(12) Patent Application Publication
(10) Pub. No.: US 2025/0143609 A1
(43) Pub. Date: May 8, 2025

(54) SYSTEMS AND METHODS FOR IMPEDIMETRIC GLUCOSE SENSING USING BORONIC ACID VIOLOGEN (OBBV)

(71) Applicant: Medtronic MiniMed, Inc., Northridge, CA (US)

(72) Inventor: Quyen B. Ong, Arcadia, CA (US)

(21) Appl. No.: 18/934,606

(22) Filed: Jan. 14, 2025

Related U.S. Application Data
(60) Provisional application No. 63/595,401, filed on Nov. 2, 2023.

(51) Int. Cl. A61B 5/1473; A61B 5/145; G01N 27/327

(52) U.S. Cl. CPC A61B 5/1473; A61B 5/14532; A61B 5/14542; G01N 27/3275

(57) ABSTRACT

An oxygen-independent analyte sensor includes at least one electrode and an oxygen-independent analyte sensing molecule disposed on the at least one electrode. The oxygen-independent analyte sensing molecule is electrografted on to the at least one electrode. The sensor may process an electrochemical impedance spectroscopy (EIS) parameter value in response to exposure to an analyte.

### CROSS-REFERENCE TO RELATED APPLICATIONS

This application claims the benefit of and priority to U.S. Provisional Patent Application Ser. No. 63/595,401 filed Nov. 2, 2023, the entire disclosure of which is incorporated by reference herein.

### FIELD

The present disclosure relates generally to continuous glucose monitoring (CGM) and more particularly to analytic sensors and methods for improving interferent rejection and longevity.

### BACKGROUND

Analyte sensors such as biosensors include devices that use biological elements to convert a chemical analyte in a matrix into a detectable signal. There are many types of biosensors used for a wide variety of analytes, including amperometric glucose sensors for glucose level control for diabetes.

A typical glucose sensor works according to the following chemical reactions:

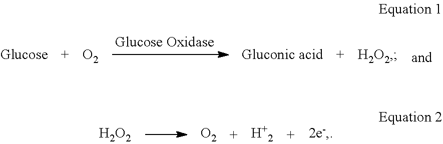

The glucose oxidase is used to catalyze the reaction between glucose and oxygen to yield gluconic acid and hydrogen peroxide, H₂O₂, (Equation 1). The hydrogen peroxide reacts electrochemically as shown in Equation 2, and the current can be measured by a potentiostat. These reactions, which occur in a variety of oxidoreductases known in the art, are used in a number of sensor designs.

One common problem with analyte sensors is that they can electrochemically react not only with the analyte to be measured (or by-product of the enzymatic reaction with the analyte), but can also react with other electroactive chemical species that are not intentionally being measured, which causes an increase in signal strength due to these “interfering species.” Typically, such interfering species are compounds with an oxidation or reduction potential that overlaps with the analyte to be measured (or a by-product of the enzymatic reaction with the analyte). For example, in a conventional amperometric glucose oxidase-based glucose sensor where the conventional sensor measures hydrogen peroxide, interfering species such as acetaminophen, ascorbate, and urate are known to confound true analyte signals, resulting in loss of sensor sensitivity or longevity.

Another common problem is that the analyte sensors need time for sensor signals to stabilize due to different sensor designs. The term “run-in” is typically used to describe the period of time needed by the sensor to stabilize during the first few days of testing/wearing of the sensor. There is room for improvement in the design of analyte sensors to reduce sensor run-in time and/or improve sensor lifetime.

### SUMMARY

The present disclosure relates to analytic sensors and methods for improving interferent rejection and longevity.

In accordance with aspects of the present disclosure, an oxygen-independent analyte sensor includes a plurality of electrodes, including a working electrode and an oxygen-independent analyte sensing molecule disposed on the working electrode. The oxygen-independent analyte sensing molecule is electrografted on to the working electrode.

In an aspect of the present disclosure, the oxygen-independent analyte sensor may further include one or more processors and one or more processor-readable media storing instructions which, when executed by the one or more processors, causes performance of: processing an electrochemical impedance spectroscopy (EIS) parameter value in response to exposure to the analyte.

In another aspect of the present disclosure, the electrode with the oxygen-independent analyte sensing molecule may be configured to generate a detectable electrical signal upon exposure to the analyte.

In yet another aspect of the present disclosure, the oxygen-independent analyte sensor may further include a competitive binding molecule.

In a further aspect of the present disclosure, the competitive binding molecule may include TriCysMA.

In yet a further aspect of the present disclosure, the instructions, when executed by the one or more processors, may further cause performance of determining based on the EIS parameter a capacitance due to double layer changes upon the oxygen-independent analyte sensing molecule binding to glucose.

In an aspect of the present disclosure, the plurality of electrodes may be configured in an interdigital arrangement.

In another aspect of the present disclosure, the oxygen-independent analyte sensing molecule may induce a change in charge transfer resistance through polarization of the plurality of electrodes upon binding to the analyte.

In yet another aspect of the present disclosure, the instructions, when executed by the one or more processors, may, during an early wear period, further cause performance of adjusting an analyte measurement based on the EIS parameter value.

In accordance with aspects of the disclosure, a processor-implemented method of determining blood glucose using an oxygen-independent analyte sensor is presented. The method includes sensing, by an oxygen-independent analyte sensor, an electrical signal in response to exposure to an analyte and determining an electrochemical impedance spectroscopy (EIS) parameter value based on the electrical signal. The sensor includes a working electrode and an oxygen-independent analyte sensing molecule electrografted on to the working electrode.

In a further aspect of the present disclosure, the method may further include determining a sensor glucose value based on the EIS parameter.

In a further aspect of the present disclosure, the electrode with the oxygen-independent analyte sensing molecule may be configured to generate a detectable electrical signal upon exposure to the analyte.

In yet a further aspect of the present disclosure, the sensor may further include a competitive binding molecule immobilized in a hydrogel embedded at an end of the analyte sensor. The competitive binding molecule may include TriCysMA.

In an aspect of the present disclosure, the method may further include determining based on the EIS parameter a capacitance due to double layer changes upon the oxygen-independent analyte sensing molecule binding to glucose.

In yet a further aspect of the present disclosure, the sensor may include a plurality of electrodes configured in an interdigital arrangement.

In an aspect of the present disclosure, the method may further include inducing a change in charge transfer resistance through polarization of the plurality of electrodes upon binding to the analyte by the oxygen-independent analyte sensing molecule.

In yet another aspect of the present disclosure, the method may further include, during an early wear period, adjusting an analyte measurement based on the EIS parameter value.

In a further aspect of the present disclosure, during the early wear period, the analyte measurement may be adjusted further based on a reference EIS parameter value.

In accordance with aspects of the disclosure, one or more non-transitory processor readable media storing instructions which, when executed by one or more processors, cause performance of: sensing, by an oxygen-independent analyte sensor, an electrical signal in response to exposure to an analyte; determining an electrochemical impedance spectroscopy (EIS) parameter value based on the electrical signal; and determining a sensor glucose value based on the EIS parameter. The sensor includes a working electrode and an oxygen-independent analyte sensing molecule disposed on the working electrode. The oxygen-independent analyte sensing molecule is electrografted on to the working electrode.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A detailed description of aspects of the disclosure will be made with reference to the accompanying drawings, wherein like numerals designate corresponding parts in the figures.

FIG. 1 illustrates a perspective view of a subcutaneous sensor insertion set and a block diagram of an analyte sensor electronics device, in accordance with one or more aspects;

FIG. 2 illustrates a substrate having a first side which contains an electrode configuration and a second side which contains electronic circuitry, in accordance with one or more aspects;

FIG. 3 illustrates a block diagram of an electronic circuit for sensing an output of an analyte sensor, in accordance with one or more aspects;

FIG. 4 illustrates a block diagram of an analyte sensor electronics device and a sensor including a plurality of electrodes, in accordance with one or more aspects;

FIG. 5 illustrates an alternative aspect including a sensor and a sensor electronics device, in accordance with one or more aspects;

FIG. 6 is a diagram of a glucose sensing molecule, in accordance with aspects of the disclosure;

FIG. 7 is a diagram of the glucose sensing molecule of FIG. 6 electrografted to an electrode of the sensor of FIG. 1, in accordance with aspects of the disclosure;

FIG. 8 illustrates a diagram of the polarization of the electrode of FIG. 7 resulting from glucose binding, in accordance with aspects of the disclosure;

FIG. 9 illustrates a diagram of a competitive binding molecule (dye) of the sensor of FIG. 1, in accordance with one or more aspects;

FIG. 10 illustrates a flowchart of an exemplary method of determining blood glucose using an oxygen-independent analyte sensor, in accordance with one or more aspects;

FIG. 11 is a graph illustrating a Bode plot for an electrode with no chemical modification subjected to 0, 100, 200, and 400 mg/dL of glucose, in accordance with one or more aspects;

FIG. 12 is a graph illustrating a Bode plot for an electrode electropolymerized with boronic acid viologen (oBBV) subjected to 0, 100, 200, and 400 mg/dL of glucose, in accordance with one or more aspects;

FIG. 13 is a graph illustrating a Bode plot for an electrode electropolymerized with oBBV with TriCysMA subjected to 0, 100, 200, and 400 mg/dL of glucose,

FIG. 14 is a graph illustrating example electrodes with and without oBBV and in presence of TriCysMA, in accordance with one or more aspects;

FIG. 15 is a graph of imaginary impedance (Z_{imag}) vs. glucose for an electrode without any chemical modifications, in accordance with one or more aspects;

FIG. 16 is a graph of Z_{imag} vs. glucose for an electrode with TriCysMA, in accordance with one or more aspects;

FIG. 17 is a graph of Z_{imag} vs. glucose for an electrode electropolymerized with oBBV, in accordance with one or more aspects;

FIG. 18 is a graph illustrating Z_{imag} vs. glucose comparison of electrodes and oBBV polymerized electrodes in modified Phosphate-Buffered Saline (PBS), in accordance with one or more aspects;

FIG. 19 is a graph illustrating Z_{imag} vs. glucose comparison of electrodes and oBBV polymerized electrodes in PBS vs in ferricyanide (FeCN), in accordance with one or more aspects; and

FIG. 20 is a graph illustrating changes in impedance at various frequencies for example oBBV polymerized electrodes, in accordance with one or more aspects.

### DETAILED DESCRIPTION

In the following description, reference is made to the accompanying drawings which form a part hereof and which illustrate several aspects of the present disclosure. It is understood that other aspects may be utilized, and structural and operational changes may be made without departing from the scope of the present disclosure.

The aspects herein are described below with reference to flowchart illustrations of methods, systems, devices, apparatus, and programming and computer program products. It will be understood that each block of the flowchart illustrations, and combinations of blocks in the flowchart illustrations, can be implemented by programming instructions, including computer program instructions (as can any menu screens described in the figures). These computer program instructions may be loaded onto a computer or other programmable data processing apparatus (such as a controller, microcontroller, or processor in a sensor electronics device) to produce a machine, such that the instructions which execute on the computer or other programmable data processing apparatus create instructions for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks, and/or menus presented herein. Programming instructions may also be stored in and/or implemented via electronic circuitry, including integrated circuits (ICs) and Application Specific Integrated Circuits (ASICs) used in conjunction with sensor devices, apparatuses, and systems.

Long run-in continuous glucose monitoring (CGM) times represent a challenge. The disclosed technology leverages a non-consumptive, oxygen-independent analyte sensor that is free of electrochemical interferents. The disclosed technology provides the benefit of improving CGM accuracy that can enable self-calibration and adjustments to day 1 run-in and provide better analyte sensor accuracy and potential to meet integrated continuous glucose monitoring (iCGM) on day 1. The term “day 1” as used herein includes the first day of use of the analyte sensor after the analyte sensor is implanted in a user.

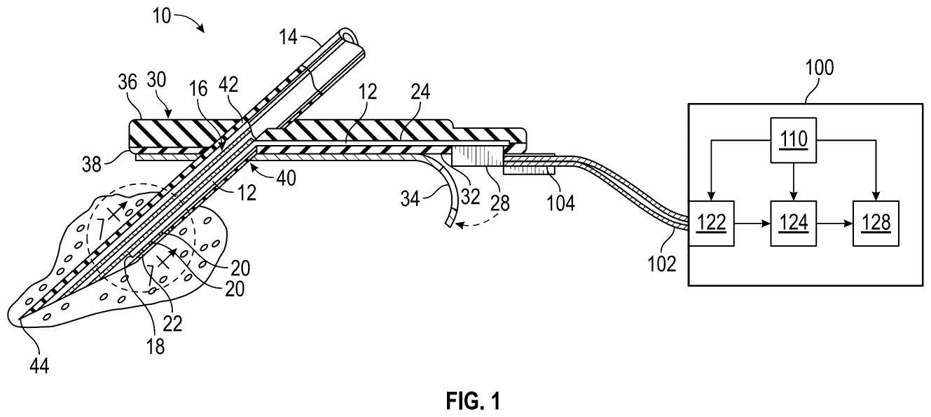

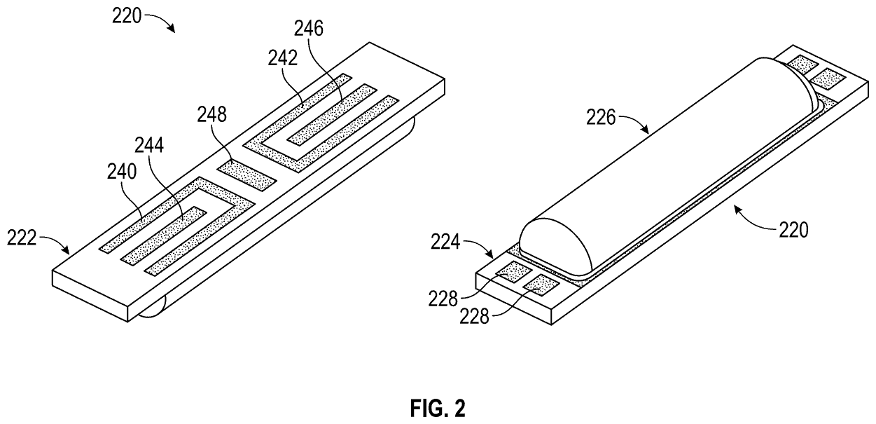

FIG. 1 is a perspective view of a subcutaneous sensor insertion set and a block diagram of a sensor electronics device according to various aspects of the disclosure. As illustrated in FIG. 1, a subcutaneous sensor set 10 is provided for subcutaneous placement of an active portion of an analyte sensor 12 (see, e.g., FIG. 2), or the like, at a selected site in the body of a user. The subcutaneous or percutaneous portion of the sensor set 10 includes a hollow, slotted insertion needle 14, and a cannula 16. The needle 14 is used to facilitate quick and easy subcutaneous placement of the cannula 16 at the subcutaneous insertion site. Inside the cannula 16 is a sensing portion 18 of the analyte sensor 12 to expose one or more sensor electrodes 20 to the user's bodily fluids through a window 22 formed in the cannula 16. In an aspect of the disclosure, the one or more sensor electrodes 20 may include a counter electrode, a reference electrode, and one or more working electrodes. After insertion, the insertion needle 14 is withdrawn to leave the cannula 16 with the sensing portion 18 and the sensor electrodes 20 in place at the selected insertion site.

In particular aspects, the subcutaneous sensor set 10 facilitates accurate placement of a flexible thin film electrochemical analyte sensor 12 of the type used for monitoring specific blood parameters representative of a user's condition. The analyte sensor 12 monitors glucose levels in the body and may be used in conjunction with automated or semi-automated medication infusion pumps of the external or implantable type as described, e.g., in U.S. Pat. Nos. 4,562,751; 4,678,408; 4,685,903 or 4,573,994, the entire contents of which are incorporated herein by reference, to control delivery of insulin to a diabetic patient.

Particular aspects of the flexible analyte sensor 12 are constructed in accordance with thin film mask techniques to include elongated thin film conductors embedded or encased between layers of a selected insulative material such as polyimide film or sheet, and membranes. The sensor electrodes 20 at a tip end of the sensing portion 18 are exposed through one of the insulative layers for direct contact with patient blood or other body fluids, when the sensing portion 18 (or active portion) of the analyte sensor 12 is subcutaneously placed at an insertion site. The sensing portion 18 is joined to a connection portion 24 that terminates in conductive contact pads, or the like, which are also exposed through one of the insulative layers. In alternative aspects, other types of implantable sensors, such as chemical based, optical based, or the like, may be used.

As is known in the art, the connection portion 24 and the contact pads are generally adapted for a direct wired electrical connection to a suitable monitor or sensor electronics device 100 for monitoring a user's condition in response to signals derived from the sensor electrodes 20. Further description of flexible thin film sensors of this general type may be found, e.g., in U.S. Pat. No. 5,391,250, which is herein incorporated by reference. The connection portion 24 may be conveniently connected electrically to the monitor or sensor electronics device 100 or by a connector block 28 (or the like) as shown and described, e.g., in U.S. Pat. No. 5,482,473, which is also herein incorporated by reference. Thus, in accordance with aspects of the present disclosure, subcutaneous sensor sets 10 may be configured or formed to work with either a wired or a wireless characteristic monitor system.

The sensor electrodes 20 may be used in a variety of sensing applications and may be configured in a variety of ways. For example, the sensor electrodes 20 may be used in physiological parameter sensing applications in which some type of biomolecule is used as a catalytic agent. For example, the sensor electrodes 20 may be used in an oxygen-independent glucose sensor.

The sensor electrodes 20, along with a biomolecule or some other catalytic agent, may be placed in a human body in a vascular or non-vascular environment. For example, the sensor electrodes 20 and biomolecule may be placed in a vein and be subjected to a blood stream, or may be placed in a subcutaneous or peritoneal region of the human body.

The monitor 100 may also be referred to as a sensor electronics device 100. The monitor 100 may include a power source 110, a sensor interface 122, processing electronics 124, and data formatting electronics 128. The monitor 100 may be coupled to the sensor set 10 by a cable 102 through a connector that is electrically coupled to the connector block 28 of the connection portion 24. In an alternative aspect, the cable 102 may be omitted. In this aspect of the disclosure, the monitor 100 may include an appropriate connector for direct connection to the connection portion 104 of the sensor set 10. The sensor set 10 may be modified to have the connector portion 104 positioned at a different location, e.g., on top of the sensor set 10 to facilitate placement of the monitor 100 over the sensor set 10.

In aspects of the disclosure, the sensor interface 122, the processing electronics 124, and the data formatting electronics 128 are formed as separate semiconductor chips, however, alternative aspects may combine the various semiconductor chips into a single or multiple customized semiconductor chips. The sensor interface 122 connects with the cable 102 that is connected with the sensor set 10.

The power source 110 may be a battery. The battery can include three series silver oxide battery cells. In alternative aspects, different battery chemistries may be utilized, such as lithium based chemistries, alkaline batteries, nickel metal hydride, or the like, and a different number of batteries may be used. The monitor 100 provides power to the sensor set via the power source 110, through the cable 102 and cable connector 104. In an aspect of the disclosure, the power is a voltage provided to the sensor set 10. In an aspect of the disclosure, the power is a current provided to the sensor set 10. In an aspect of the disclosure, the power is a voltage provided at a specific voltage to the sensor set 10.

FIG. 2 illustrates an implantable analyte sensor and electronics for driving the implantable analyte sensor according to an aspect of the present disclosure. FIG. 2 shows a substrate or flex 220 having two sides; a first side 222 which contains an electrode configuration and a second side 224 of which contains electronic circuitry. As in FIG. 2, the first side 222 of the substrate includes two counter electrode-working electrode pairs 240, 242, 244, 246 on opposite sides of a reference electrode 248. A second side 224 of the substrate includes electronic circuitry. As shown, the electronic circuitry may be enclosed in a hermetically sealed casing 226, providing a protective housing for the electronic circuitry. This allows the sensor substrate 220 to be inserted into a vascular environment or other environment which may subject the electronic circuitry to fluids. By sealing the electronic circuitry in a hermetically sealed casing 226, the electronic circuitry may operate without risk of short circuiting by the surrounding fluids. Also shown in FIG. 2, pads 228 are connected to the input and output lines of the electronic circuitry. The electronic circuitry itself may be fabricated in a variety of ways. According to an aspect of the present disclosure, the electronic circuitry may be fabricated as an integrated circuit using techniques common in the industry.

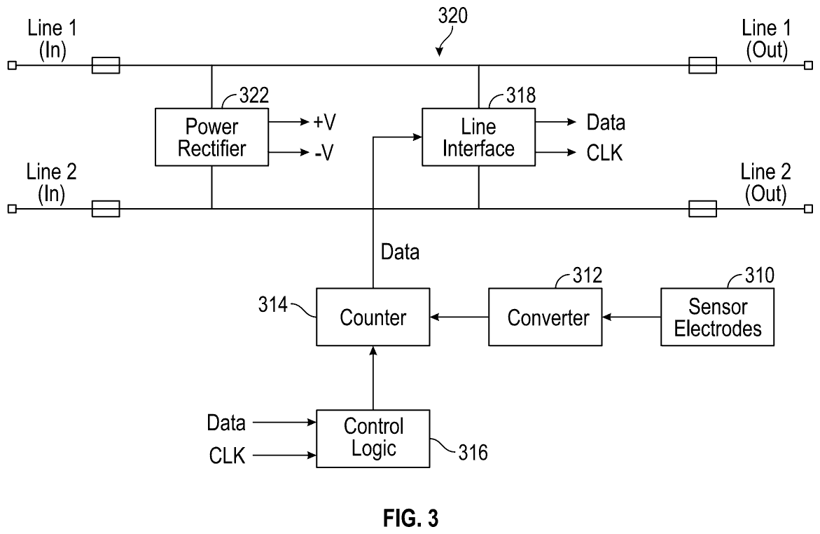

FIG. 3 illustrates a general block diagram of an electronic circuit for sensing an output of an analyte sensor according to aspects of the present disclosure. At least one pair of sensor electrodes 310 may interface to a data converter 312, the output of which may interface to a counter 314. The counter 314 may be controlled by control logic 316. The output of the counter 314 may connect to a line interface 318. The line interface 318 may be connected to input and output lines 320 and may also connect to the control logic 316. The input and output lines 320 may also be connected to a power rectifier 322.

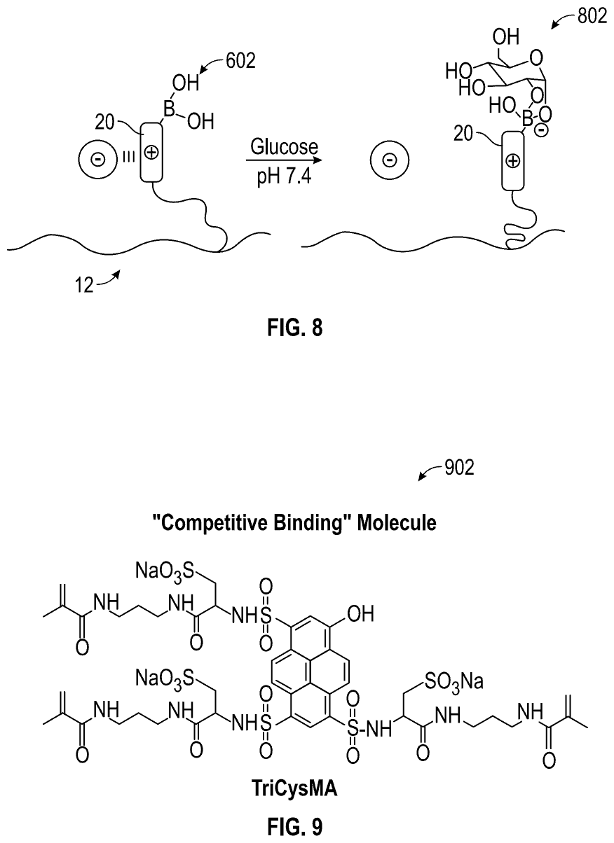

The sensor electrodes 310 may be used in a variety of sensing applications and may be configured in a variety of ways. For example, the sensor electrodes 310 may be used in physiological parameter sensing applications in which some type of biomolecule is used as a catalytic agent. For example, the sensor electrodes 310 may be used in an oxygen-independent glucose sensor having Boronic acid viologen (oBBV) that binds with the glucose. The sensor electrodes 310, along with oBBV and/or a dye (FIG. 8), may be placed in a human body in a vascular or non-vascular environment. For example, the sensor electrodes 310 and biomolecule may be placed in a vein and be subjected to a blood stream.

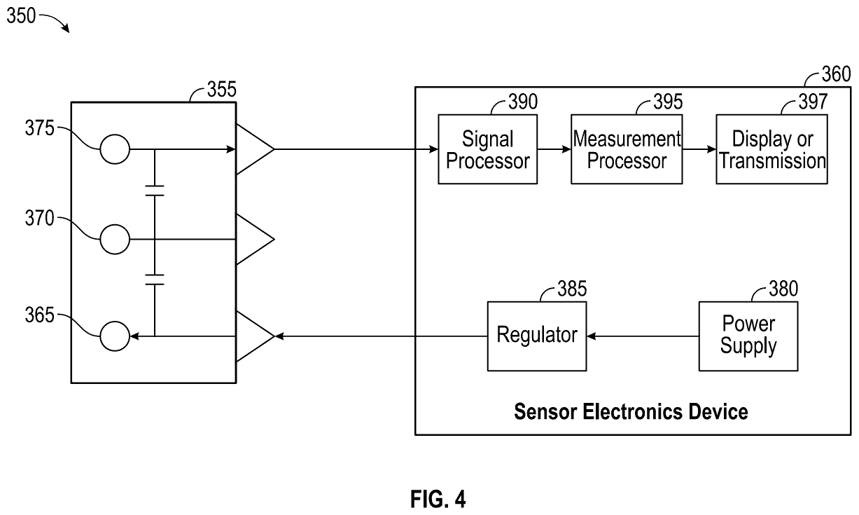

FIG. 4 illustrates a block diagram of an analyte sensor electronics device and a sensor including a plurality of electrodes according to an aspect of the disclosure. The sensor set or system 350 includes an analyte sensor 355 and a sensor electronics device 360. The analyte sensor 355 includes a counter electrode 365, a reference electrode 370, and a working electrode 375. The sensor electronics device 360 includes a power supply 380, a regulator 385, a signal processor 390, a measurement processor 395, and a display/transmission module 397. The power supply 380 provides power (in the form of either a voltage, a current, or a voltage including a current) to the regulator 385. The regulator 385 transmits a regulated voltage to the analyte sensor 355. In an aspect of the disclosure, the regulator 385 transmits a voltage to the counter electrode 365 of the analyte sensor 355.

The analyte sensor 355 creates a sensor signal indicative of a concentration of a physiological characteristic being measured. For example, the sensor signal may be indicative of a blood glucose reading. The sensor signal may be measured at the working electrode 375. In an aspect of the disclosure, the sensor signal may be a current measured at the working electrode. In an aspect of the disclosure, the sensor signal may be a voltage measured at the working electrode.

The signal processor 390 receives the sensor signal (e.g., a measured current or voltage) after the sensor signal is measured at the analyte sensor 355 (e.g., the working electrode). The signal processor 390 processes the sensor signal and generates a processed sensor signal. The measurement processor 395 receives the processed sensor signal and calibrates the processed sensor signal utilizing reference values. In an aspect of the disclosure, the reference values are stored in a reference memory and provided to the measurement processor 395. The measurement processor 395 generates sensor measurements. The sensor measurements may be stored in a measurement memory (not shown). The sensor measurements may be sent to a display/transmission device to be either displayed on a display in a housing with the sensor electronics or transmitted to an external device.

The sensor electronics device 360 may be a monitor which includes a display to display physiological characteristics readings. The sensor electronics device 360 may also be installed in a desktop computer, a pager, a television including communications capabilities, a laptop computer, a server, a network computer, a personal digital assistant (PDA), a portable telephone including computer functions, an infusion pump including a display, and/or a combination infusion pump/analyte sensor. The sensor electronics device 360 may be housed in a cellular phone, a smartphone, a network device, a home network device, and/or other appliance connected to a home network.

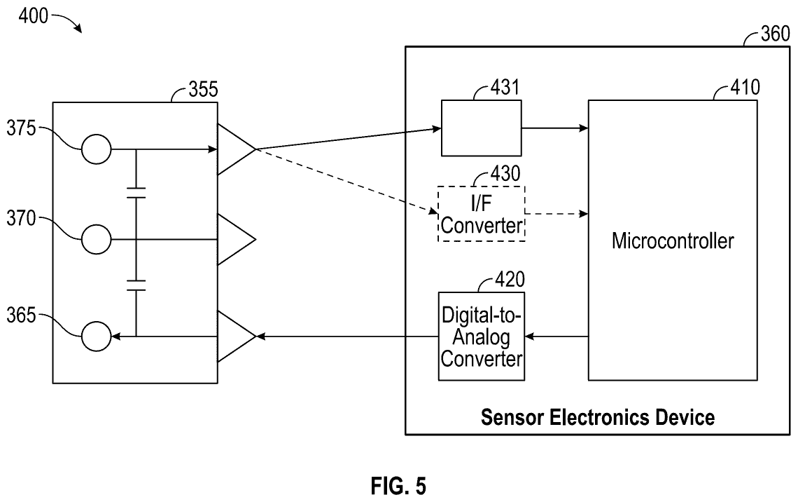

FIG. 5 illustrates an alternative aspect including an analyte sensor and a sensor electronics device according to an aspect of the present disclosure. The sensor set or sensor system 400 includes the sensor electronics device 360 and the analyte sensor 355. The analyte sensor 355 includes the counter electrode 365, the reference electrode 370, and the working electrode 375. The sensor electronics device 360 includes a microcontroller 410 and a digital-to-analog converter (DAC) 420. The sensor electronics device 360 may also include a current-to-frequency converter (I/F converter) 430.

The microcontroller 410 includes software program code or programmable logic which, when executed, causes the microcontroller 410 to transmit a signal to the DAC 420, where the signal is representative of a voltage level or value that is to be applied to the analyte sensor 355. The DAC 420 receives the signal and generates the voltage value at the level instructed by the microcontroller 410. In aspects of the disclosure, the microcontroller 410 may change the representation of the voltage level in the signal frequently or infrequently. Illustratively, the signal from the microcontroller 410 may instruct the DAC 420 to apply a first voltage value for one second and a second voltage value for two seconds.

The analyte sensor 355 may receive the voltage level or value. In an aspect of the disclosure, the counter electrode 365 may receive the output of an operational amplifier which has as inputs the reference voltage and the voltage value from the DAC 420. The application of the voltage level causes the analyte sensor 355 to create a sensor signal indicative of a concentration of a physiological characteristic being measured. In an aspect of the disclosure, the microcontroller 410 may measure the sensor signal (e.g., a current value) from the working electrode. Illustratively, a sensor signal measurement circuit 431 may measure the sensor signal. In an aspect of the disclosure, the sensor signal measurement circuit 431 may include a resistor and the current may be passed through the resistor to measure the value of the sensor signal. In an aspect of the disclosure, the sensor signal may be a current level signal and the sensor signal measurement circuit 431 may be a current-to-frequency (I/F) converter 430. The I/F converter 430 may measure the sensor signal in terms of a current reading, convert it to a frequency-based sensor signal or electrochemical impedance spectroscopy (“EIS”) signal, and transmit the frequency-based sensor signal or EIS signal to the microcontroller 410. Persons skilled in the art will understand how to implement and apply EIS. Various aspects of EIS signals are described in U.S. Patent Application Publication No. US2013/0060105A1, which is hereby incorporated by reference herein in its entirety. In aspects of the disclosure, the microcontroller 410 may be able to receive frequency-based sensor signals easier than non-frequency-based sensor signals. The microcontroller 410 receives the sensor signal, whether frequency-based or non-frequency-based, and determines a value for the physiological characteristic of a subject, such as a blood glucose level. The microcontroller 410 may include program code, which when executed or run, is able to receive the sensor signal and convert the sensor signal to a physiological characteristic value.

In one aspect of the disclosure, the microcontroller 410 may convert the sensor signal to a blood glucose level. While converting the sensor signal to a blood glucose value, the microcontroller 410 may use one or more models, which are specific ways to use the sensor signal to calculate the blood glucose value. In some aspects, the microcontroller 410 may utilize measurements (e.g., sensor signals and electrochemical impedance spectroscopy (EIS) signals from the analyte sensor 355) stored within an internal memory in order to determine the blood glucose level of the subject. In some aspects, the microcontroller 410 may utilize measurements stored within a memory external to the microcontroller 410 to assist in determining the blood glucose level of the subject.

After the physiological characteristic value is determined by the microcontroller 410, the microcontroller 410 may store measurements of the physiological characteristic values for a number of time periods. For example, a blood glucose value (BG) may be sent to the microcontroller 410 from the sensor every second or five seconds, and the microcontroller may save sensor measurements for five minutes or ten minutes of BG readings. The microcontroller 410 may transfer the measurements of the physiological characteristic values to a display on the sensor electronics device 360. For example, the sensor electronics device 360 may be a monitor which includes a display that provides a blood glucose reading for a subject. In one aspect of the disclosure, the microcontroller 410 may transfer the measurements of the physiological characteristic values to an output interface of the microcontroller 410. The output interface of the microcontroller 410 may transfer the measurements of the physiological characteristic values, e.g., blood glucose values, to an external device, e.g., an infusion pump, a combined infusion pump/glucose meter, a computer, a personal digital assistant, a pager, a network appliance, a server, a cellular phone, or any computing device.

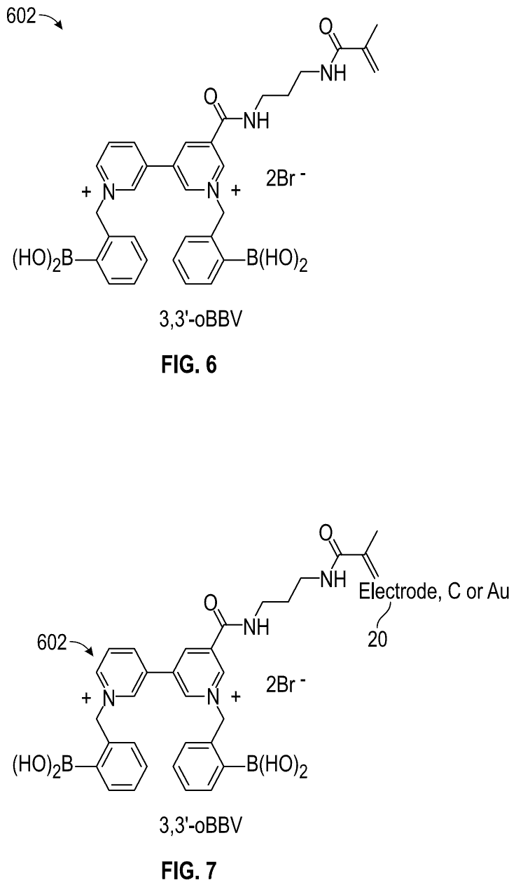

FIG. 6 is a diagram illustrating the structure of an example oxygen-independent analyte sensing molecule 602 of oxygen-independent analyte sensor 355 (FIG. 4). Boronic acid viologen (oBBV) may be used to bind to analytes such as glucose, to provide an oxygen-independent analyte sensor. In one example, the boronic acid is covalently bonded to a conjugated nitrogen-containing heterocyclic aromatic bis-onium structure (e.g., a viologen). “Viologen” refers generally to compounds having the basic structure of a nitrogen-containing conjugated N-substituted heterocyclic aromatic bis-onium salt, such as 2,2′-, 3,3′- or 4,4′-N,N′ bis-(benzyl) bipyridium dihalide (e.g., dichloride, bromide chloride). In aspects, polyviologens may be used to bind to analytes such as glucose, to provide an oxygen-independent analyte sensor.

In aspects, the oxygen-independent analyte sensing molecule 602 may include any of the following structures:

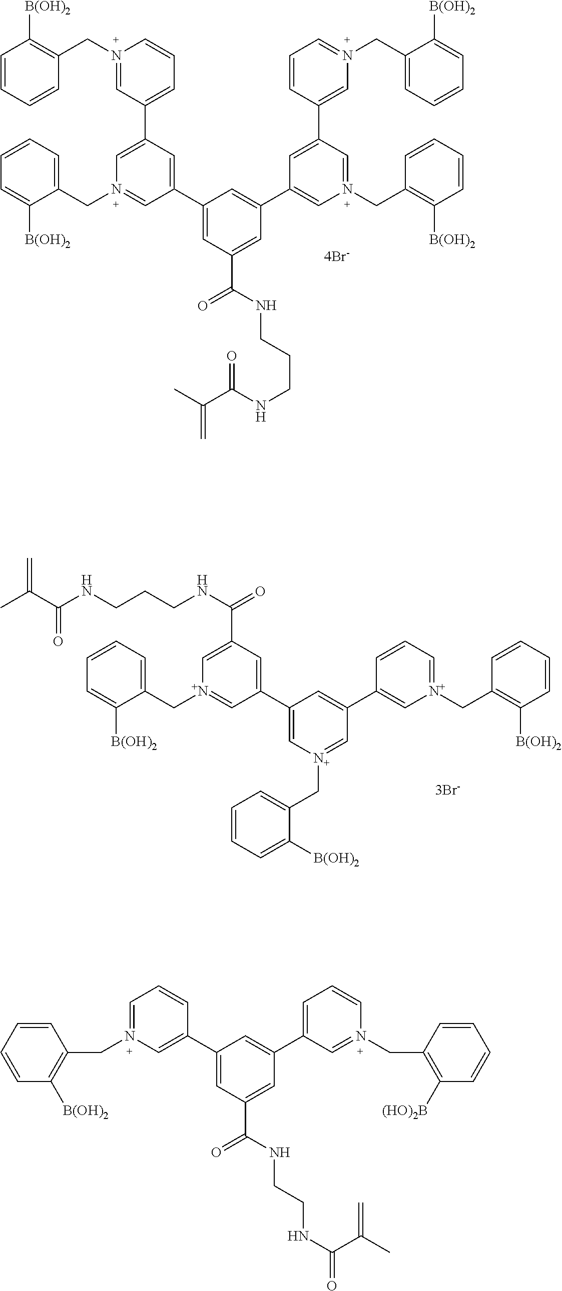

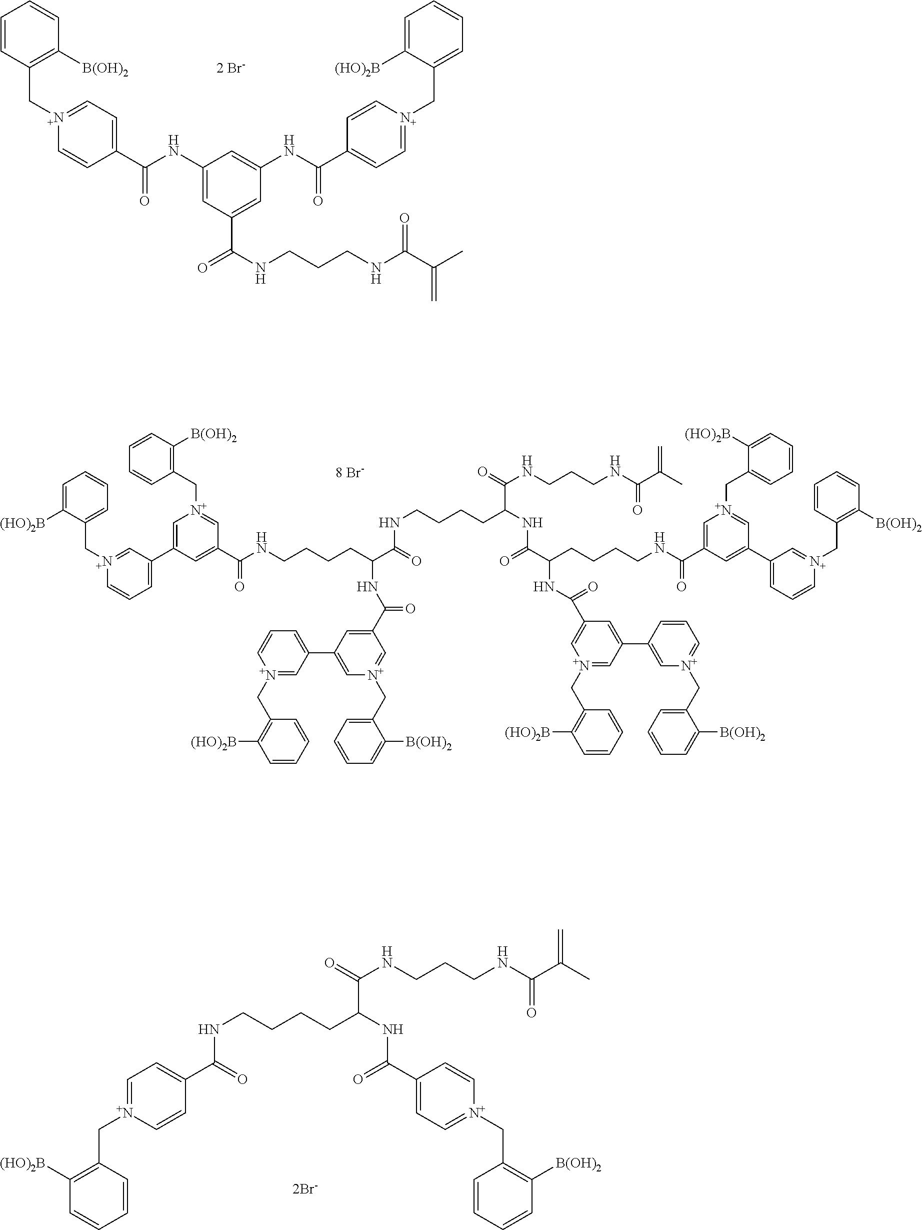

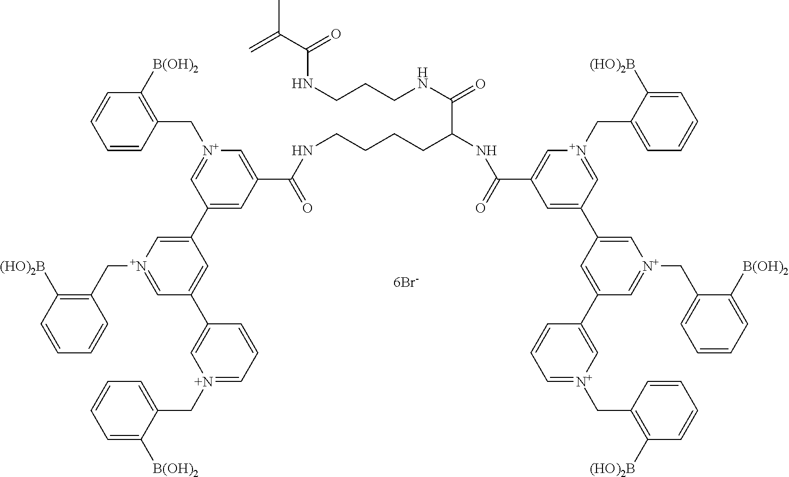 [SMILES: C=C(C)C(=O)NCCCNC(=O)C(CCCCNC(=O)c1cc(-c2cc(-c3ccc[n+](Cc4ccccc4B(O)O)c3)c[n+](Cc3ccccc3B(O)O)c2)c[n+](Cc2ccccc2B(O)O)c1)NC(=O)c1cc(-c2cc(-c3ccc[n+](Cc4ccccc4B(O)O)c3)c[n+](Cc3ccccc3B(O)O)c2)c[n+](Cc2ccccc2B(O)O)c1]

FIG. 7 is a diagram of the oxygen-independent analyte sensing molecule 602 electropolymerized and/or electrografted to an electrode 20 of the analyte sensor 355 of FIG. 4. The electrode 20 with the oxygen-independent analyte sensing molecule 602 is configured to generate an electrical signal (e.g., an EIS signal) in response to the binding of glucose to the oxygen-independent analyte sensing molecule 602. The electrical signal may be used to determine one or more EIS parameter values. Pulse voltammetry or cyclic voltammetry may be used for electrografting the oxygen-independent analyte sensing molecule 602 to the electrode(s). For example, the oxygen-independent analyte sensing molecule 602 may be electropolymerized to the electrode 20 under the following example conditions: a 10 mL aqueous solution containing approximately 0.1M oBBV and about 0.1 mM ZnCl2 to produce a polymeric layer on the gold surface of the electrode 20. Next, the electropolymerization may be carried out at a pulsed potential of about −1.297 volts vs. an AgCl reference electrode for about 1 second and repeated approximately every 1 minute for 15 times. In order to strip any Zn crystals that may have formed on the electrode surface, −0.603 volts may be imposed on the gold electrode for about for about 1 second, and repeated approximately every 1 minute for about 5 times. While the electrodes 20 are described using gold (Au), other types of electrodes are contemplated, such as carbon-based screen-printed electrodes (CSPE) and/or platinum.

FIG. 8 is a diagram illustrating the polarization of electrode 20 of FIG. 7 resulting from analyte (e.g., glucose) binding. A change in charge transfer resistance through polarization of the electrode 20 is induced upon binding to the analyte such as glucose 802. When an analyte such as glucose binds to the oxygen-independent analyte sensing molecule 602, charge transfer resistance (Rct) and/or double layer capacitance (Cd) of the analyte sensor changes, which provides changes in the EIS signal.

FIG. 9 illustrates a diagram of the structure of an example competitive binding molecule 902 (dye) of the analyte sensor 355 of FIG. 4. The competitive binding molecule 902 may be immobilized in a hydrogel embedded on the electrodes 20 of the analyte sensor 355 or may be electropolymerized to the electrodes 20. The competitive binding molecule 902 may include, for example, a polymerized TriCysMA. In this case, the competitive binding molecule 902 (TriCysMA) is not used for its florescent properties when bound to glucose. Instead, the competitive binding molecule 902 dissociates from the analyte binding molecule upon binding of the analyte binding molecule to glucose. For example, the competitive binding molecule 902 may provide more accurate EIS parameter values. The competitive binding molecule 902 may be one of any molecules carrying multiple negatively charged functionalities such as a sulfonic acid or carboxylic acid group, in addition it also contains an acrylamide moiety to facilitate either electropolymerization to the electrode to grafting to a hydrogel construct. Although TriCysMA is used as an example, the use of any negatively charged molecules with an acrylamide functionality is contemplated, for example, 3-Sulfopropyl methacrylate. The competitive binding molecule 902 may include, for example, 2-acrylamido-2-methylpropane sulfonic acid (AMPS) and/or Acrylic Acid-2-Acrylamido-2-Methylpropane Sulfonic Acid Copolymer (AA-AMPS). In aspects, the competitive binding molecule 902 may include, for example, Mono-CysMA or Bis-CysMA.

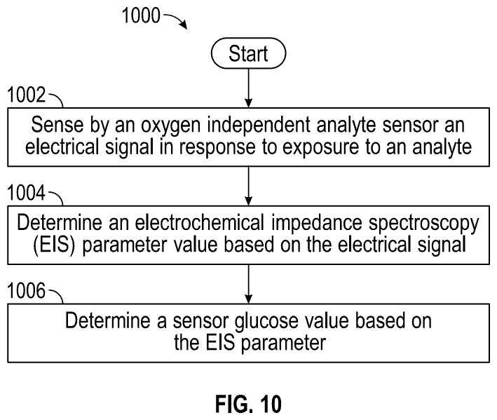

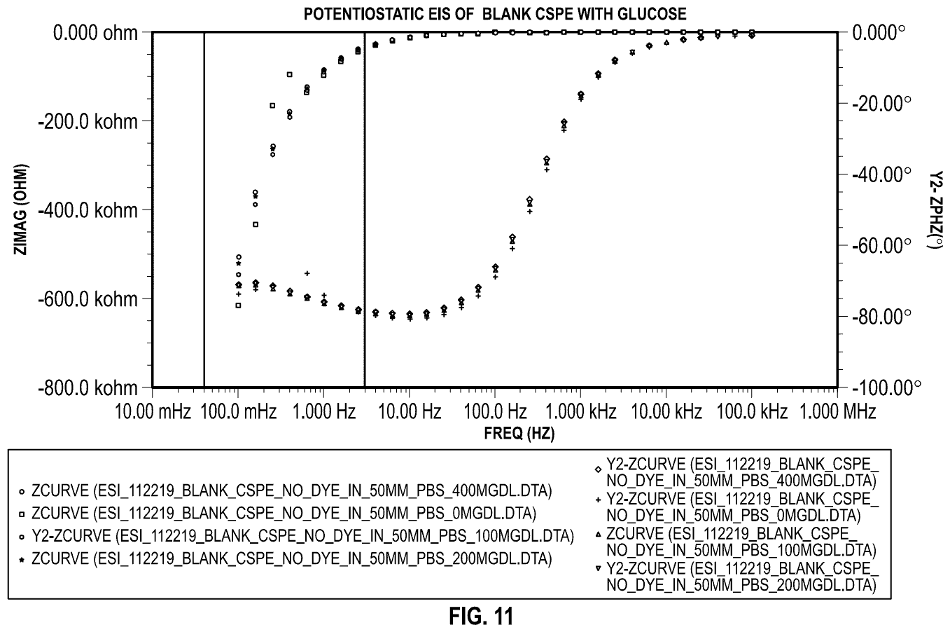

FIG. 10 is a diagram illustrating an exemplary method 1000 for determining blood glucose using an oxygen-independent analyte sensor 355 of FIG. 8. The oxygen-independent analyte sensor 355 is implanted in the body of a user and is configured to generate real-time data relating to glucose sensitivity. The analyte signal (e.g., glucose signal) is substantially comprised of a signal contribution from the analyte. The oxygen-independent analyte sensor 355 includes one or more electrodes 20 and an oxygen-independent analyte sensing molecule 602 polymerized on to the one or more electrodes. The electrodes 20 may be configured in an interdigital arrangement (FIG. 2). In aspects, the oxygen-independent analyte sensing molecule 602 may be electrografted on to the one or more electrodes 20. The oxygen-independent analyte sensing molecule 602 may include oBBV. In aspects, the oxygen-independent analyte sensor 355 may be used in an orthogonally redundant glucose sensor. The oxygen-independent analyte sensing molecule 602 induces a change in charge transfer resistance through polarization of the electrode(s) 20 upon binding to the analyte (e.g., glucose). Although glucose is used as an example, other analytes are contemplated to be within the scope of the disclosure.

At step 1002, an electrical signal (e.g., an EIS signal) is sensed by the oxygen-independent analyte sensor 355 in response to exposure to the analyte (e.g., glucose). The oxygen-independent analyte sensor 355 may further include a competitive binding molecule 902 for enhancing the linearity of the sensed electrical signal. In aspects, the competitive binding molecule 902 can be confined by a semi-permeable membrane that allows the passage of the analyte but blocks the passage of the sensing moieties. In aspects, the competitive binding molecule 902 and the oxygen-independent analyte sensing molecule 602 are operably coupled to each other.

At step 1004, EIS signature values of the oxygen-independent analyte sensor 355 are determined based on the electrical signal. As described above, the EIS signature values may be computed based on the EIS signal. A microcontroller (e.g., microcontroller 410) of the oxygen-independent analyte sensor 355 may receive an electrochemical impedance spectroscopy (“EIS”) signal from one or more working electrodes of the oxygen-independent analyte sensor 355 and calculate real impedance (Zᵣₑₐₗ) and imaginary impedance (Z_{imag}) of the EIS signal.

At step 1006, a sensor glucose (SG) value is determined based on the EIS parameter value (e.g., Z_{imag}). The SG value may be displayed, communicated to another device, and/or used by an algorithm for further processing.

In aspects, during an early wear period, an analyte measurement may be adjusted based on the EIS parameter value. During the early wear period, the analyte measurement may be adjusted further based on a reference EIS parameter value.

In aspects, the oxygen-independent analyte sensor 355 may be combined with a glucose oxidase (Gox) based sensor. For example, the oxygen-independent analyte sensor 355 may be used for the internal calibration of a Gox based sensor further enabling calibration free glucose sensing, and correction for response to acetaminophen (Gox sensor). For example, the oxygen-independent analyte sensor 355 may be used to calibrate and mitigate sensitivity loss of the Gox based sensor.

The disclosed technology provides the benefit of being acetaminophen and ascorbic acid interference free, thereby minimizing such challenges.

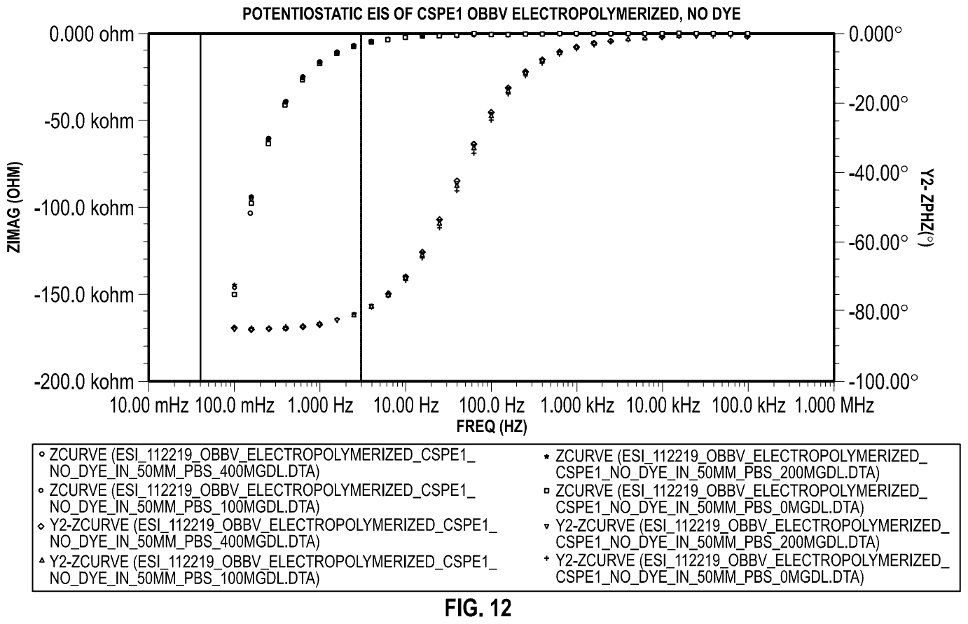

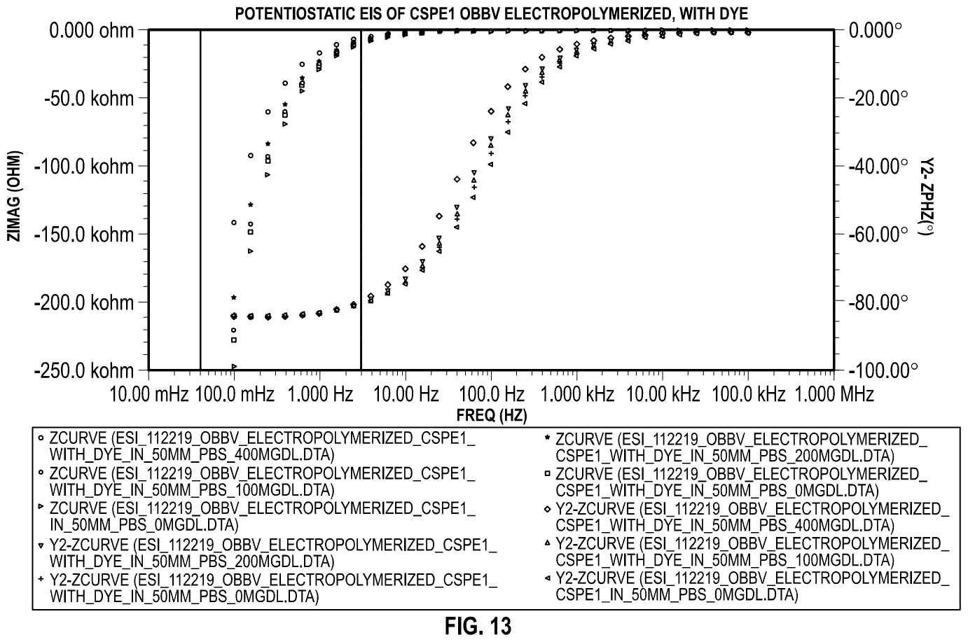

FIGS. 11-13 are graphs illustrating Bode plots for electrodes in solutions with various amounts of glucose. In FIG. 11 the electrode (without oBBV and/or dye) is subjected to 0, 100, 200, and 400 mg/dL of glucose. In FIG. 12 the electrode is electropolymerized with oxygen-independent analyte sensing molecule 602 (oBBV) and is subjected to 0, 100, 200, and 400 mg/dL of glucose. The results shown for the electrode electropolymerized with oBBV (FIG. 12) are more linear than the unmodified electrode (FIG. 11). In FIG. 13 the electrode is electropolymerized with oBBV and with the competitive binding molecule 902 (dye) and is subjected to 0, 100, 200, and 400 mg/dL of glucose. As can be seen from the graphs, the dye helps to linearize and increase the magnitude of the results further.

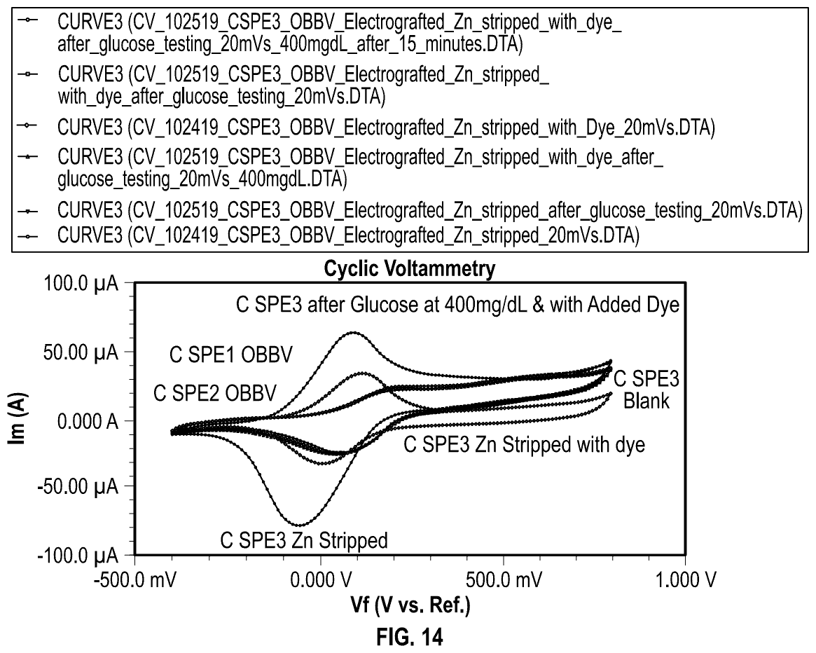

FIG. 14 is a graph illustrating example electrodes with and without the oxygen-independent analyte sensing molecule 602 at various stages in the process. Electrode 1 (CSPE1) is electrografted with oBBV by pulse voltammetry and electrodes 2 (CSPE2) and 3 (CSPE3) are electrografted with oBBV by pulse voltammetry. The graph also shows electrode 3 (CSPE3 Blank) prior to being electrografted with oBBV as well as electrografted electrode 3 in a glucose solution with dye (CSPE3 after glucose at 400 mg/dL with added dye). The plots of the electrografted electrodes have the zinc (Zn) stripped.

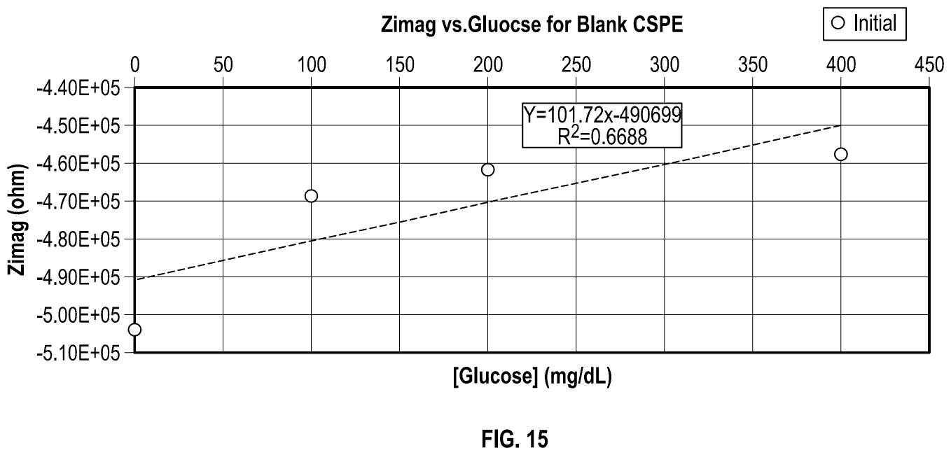

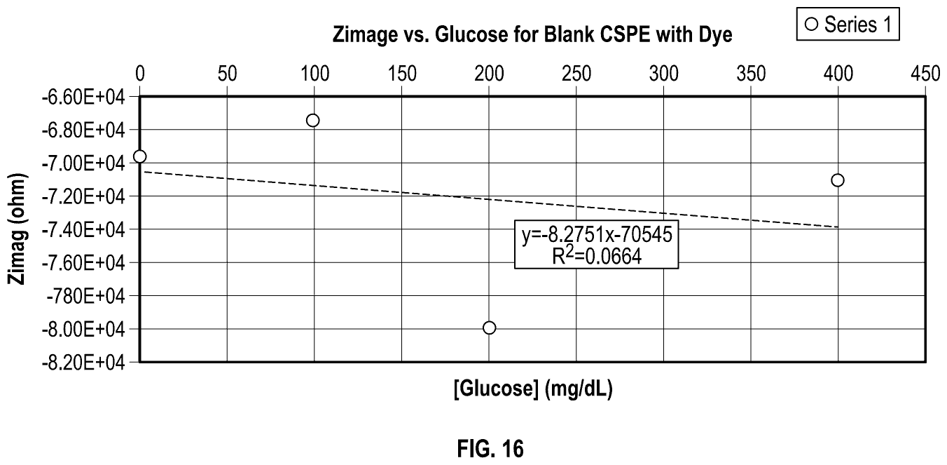

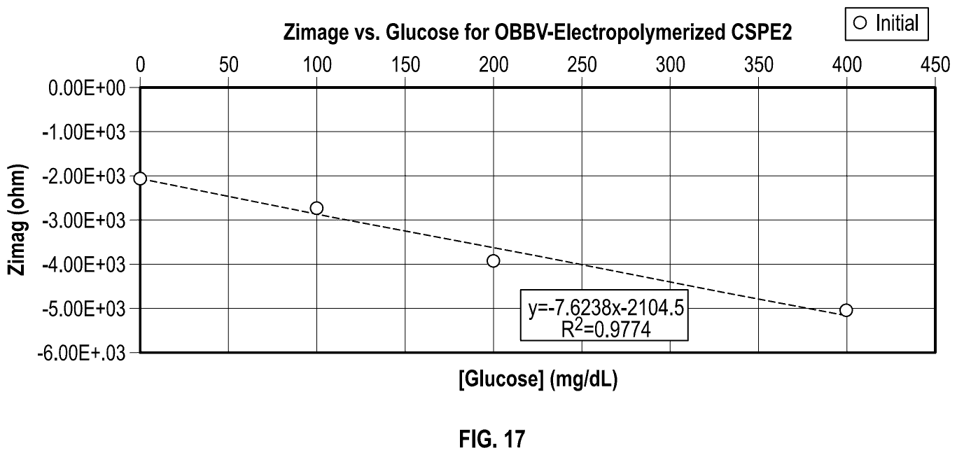

FIGS. 15-17 are comparison plots of imaginary impedance (Z_{imag}) (ohms) vs. glucose (mg/dL) for an electrode without dye (FIG. 15), for an electrode with dye (FIG. 16), and for an electrode electropolymerized with oBBV (FIG. 17). Impedimetric glucose response may be observed (at higher frequencies) where Z_{imag} trended more negative with increasing glucose concentration, in contrast with blank control (i.e., Z_{imag} shows more +ve). FIG. 17 illustrates that good linearity (R2=97%) is observed for the electrode with oBBV (oBBV CSPE2).

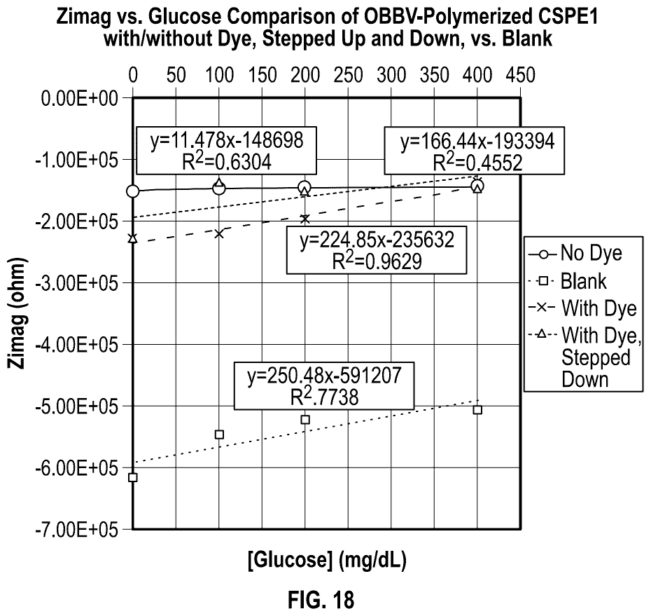

FIG. 18 is a graph illustrating an imaginary impedance (Z_{imag}) (ohms) vs. glucose (mg/dL) comparison of electrodes and oBBV electropolymerized electrodes in a Phosphate-Buffered Saline (PBS). The data is significantly more linear and higher in magnitude in the presence of the competitive binding molecule 902 than without. The magnitude of the EIS response is significantly higher for oBBV electrografted electrodes vs. non-electrografted (i.e., without oBBV). The graph illustrates that the response of the sensor electrodes to glucose provides excellent linearity.

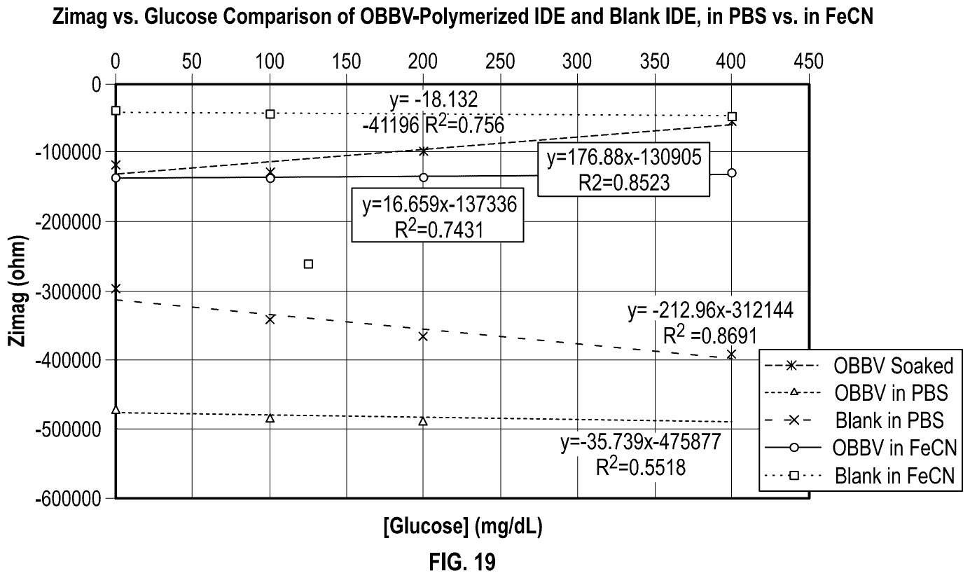

FIG. 19 is a graph illustrating imaginary impedance (Z_{imag}) vs. glucose comparison of electrodes and oBBV polymerized electrodes in PBS vs in 5 mm ferricyanide (FeCN). FeCN is negatively charged and negates charge polarization of the electrode resulting from displacement of dye through binding to glucose. The graph shows no glucose response for oBBV electrografted electrodes. This graph reinforces the concept of charge polarization in PBS, leading to observed significant differences in glucose response in FeCN vs. PBS.

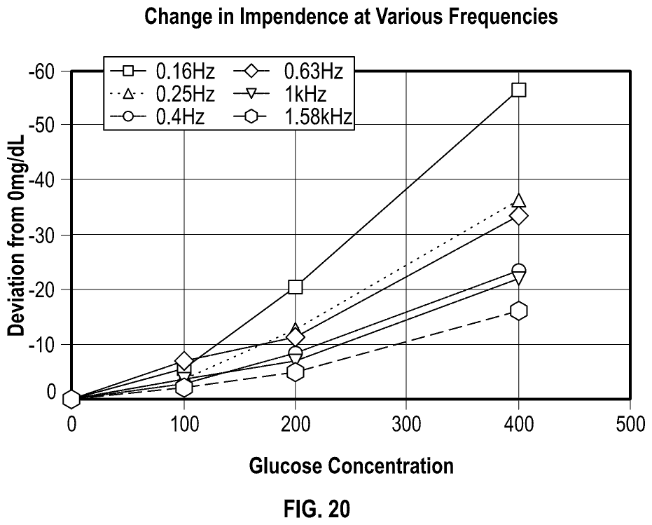

FIG. 20 is a graph illustrating changes in impedance at various frequencies for an example oBBV polymerized electrodes. The frequency is swept from about 0.16 Hz to about 1.58 kHz.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, the above-described servers and computing devices.

While the description above refers to particular aspects of the present disclosure, it will be understood that many modifications may be made without departing from the spirit thereof. Additional steps and changes to the order of the algorithms can be made while still performing the key teachings of the present disclosure. Thus, the accompanying claims are intended to cover such modifications as would fall within the true scope and spirit of the present disclosure. The presently disclosed aspects are, therefore, to be considered in all respects as illustrative and not restrictive, the scope of the disclosure being indicated by the appended claims rather than the foregoing description. Unless the context indicates otherwise, any aspect disclosed herein may be combined with any other aspect or aspects disclosed herein. All changes that come within the meaning of, and range of, equivalency of the claims are intended to be embraced therein.

What is claimed is:

1. An oxygen-independent analyte sensor comprising:
at least one electrode; and
an oxygen-independent analyte sensing molecule disposed on the at least one electrode, wherein the at least one electrode is electropolymerized with the oxygen-independent analyte sensing molecule.

2. The analyte sensor of claim 1, further comprising:
one or more processors; and
one or more processor-readable media storing instructions which, when executed by the one or more processors, causes performance of:
processing an electrochemical impedance spectroscopy (EIS) parameter value in response to exposure to the analyte.

3. The analyte sensor of claim 1, wherein the at least one electrode with the oxygen-independent analyte sensing molecule is configured to generate a detectable electrical signal upon exposure to the analyte.

4. The analyte sensor of claim 1, further comprising a competitive binding molecule.

5. The analyte sensor of claim 4, wherein the competitive binding molecule includes TriCysMA.

6. The analyte sensor of claim 2, wherein the instructions, when executed by the one or more processors, further causes performance of:
determining, based on the EIS parameter, a capacitance due to double layer changes upon the oxygen-independent analyte sensing molecule binding to glucose.

7. The analyte sensor of claim 1, wherein the at least one electrode includes a plurality of electrodes configured in an interdigital arrangement.

8. The analyte sensor of claim 1, wherein the oxygen-independent analyte sensing molecule induces a change in charge transfer resistance through polarization of the at least one electrode upon binding to the analyte.

9. The analyte sensor of claim 2, wherein the instructions, when executed by the one or more processors, further causes performance of:
during an early wear period, adjusting an analyte measurement based on the EIS parameter value.

10. The analyte sensor of claim 9, wherein, during the early wear period, the analyte measurement is adjusted further based on a reference EIS parameter value.

11. A processor-implemented method of determining blood glucose using an oxygen-independent analyte sensor, the method comprising:
sensing, by an oxygen-independent analyte sensor, an electrical signal in response to exposure to an analyte, the oxygen-independent analyte sensor including a working electrode and an oxygen-independent analyte sensing molecule disposed on the working electrode, wherein the oxygen-independent analyte sensing molecule is electrografted on to the working electrode; and
determining an electrochemical impedance spectroscopy (EIS) parameter value based on the electrical signal.

12. The processor-implemented method of claim 11, further comprising determining a sensor glucose value based on the EIS parameter.

13. The processor-implemented method of claim 11, wherein the working electrode with the oxygen-independent analyte sensing molecule is configured to generate a detectable electrical signal upon exposure to the analyte.

14. The processor-implemented method of claim 13, wherein the sensor further includes a competitive binding molecule immobilized in a hydrogel embedded at an end of the analyte sensor, and the competitive binding molecule includes TriCysMA.

15. The processor-implemented method of claim 11, further comprising determining based on the EIS parameter a capacitance due to double layer changes upon the oxygen-independent analyte sensing molecule binding to glucose.

16. The processor-implemented method of claim 11, wherein the at least one electrode includes a plurality of electrodes configured in an interdigital arrangement.

17. The processor-implemented method of claim 11, further comprising inducing a change in charge transfer resistance through polarization of the plurality of electrodes upon binding to the analyte by the oxygen-independent analyte sensing molecule.

18. The processor-implemented method of claim 11, further comprising, during an early wear period, adjusting an analyte measurement based on the EIS parameter value.

19. The processor-implemented method of claim 18, wherein, during the early wear period, the analyte measurement is adjusted further based on a reference EIS parameter value.

20. One or more non-transitory processor-readable media storing instructions which, when executed by one or more processors, cause performance of:
sensing, by an oxygen-independent analyte sensor, an electrical signal in response to exposure to an analyte, the sensor including a working electrode and an oxygen-independent analyte sensing molecule disposed on the working electrode, wherein the oxygen-independent analyte sensing molecule is electrografted on to the working electrode;
determining an electrochemical impedance spectroscopy (EIS) parameter value based on the electrical signal; and
determining a sensor glucose value based on the EIS parameter.
